(19) United States
(12) Patent Application Publication
(10) Pub. No.: US 2022/0039872 A1
(43) Pub. Date: Feb. 10, 2022

(54) Automated Endoscope Length Detection

(71) Applicant: Asensus Surgical US, Inc., Durham, NC (US)

(72) Inventors: Zachary S. Leonard, Raleigh, NC (US); William Richard Pierpont, Morrisville, NC (US)

(21) Appl. No.: 17/368,762

(22) Filed: Jul. 6, 2021

Related U.S. Application Data
(60) Provisional application No. 63/048,176, filed on Jul. 5, 2020.

(51) Int. Cl. A61B 34/20; A61B 34/30; A61B 1/313; A61B 17/34

(52) U.S. Cl. CPC A61B 34/20; A61B 34/30; A61B 2034/302; A61B 17/34; A61B 2034/2065; A61B 1/3132

(57) ABSTRACT

The length of an endoscope carried by a robotic arm is determined using computer vision. The endoscope is inserted through a trocar into a body cavity and mounted to a manipulator arm. The position of a fulcrum for movement of the endoscope at the trocar site is determined using input from a force/torque sensor. While images are captured using the endoscope, the manipulator arm withdraws the distal end of the endoscope intro the trocar. Image processing is used to determine when the distal end of the trocar becomes visible in the captured images. The position of the endoscope at the point where the trocar becomes visible is recorded, and the length of the endoscope is determined based on the recorded position.

### BACKGROUND

There are various types of surgical robotic systems on the market or under development. Some surgical robotic systems use a plurality of robotic arms. Each arm carries a surgical instrument, or the camera (also referred to in this application as the “scope” or “endoscope”) used to capture images from within the body for display on a monitor. Other surgical robotic systems use a single arm that carries a plurality of instruments and a camera that extend into the body via a single incision. Each of these types of robotic systems uses motors to position and/or orient the camera and instruments and to, where applicable, actuate the instruments. Typical configurations allow two or three instruments and the camera to be supported and manipulated by the system. Input to the system is generated based on input from a surgeon positioned at a surgeon console, typically using input devices such as input handles and a foot pedal. Motion and actuation of the surgical instruments and the camera is controlled based on the user input. The image captured by the camera is shown on a display at the surgeon console. The console may be located patient-side, within the sterile field, or outside of the sterile field.

US Patent Publication US 2010/0094312 describes a surgical robotic system in which sensors are used to determine the forces that are being applied to the patient by the robotic surgical tools during use. This application describes the use of a 6 DOF force/torque sensor attached to a surgical robotic manipulator as a method for determining the haptic information needed to provide force feedback to the surgeon at the user interface. It describes a method of force estimation and a minimally invasive medical system, in particular a laparoscopic system, adapted to perform this method. As described, a robotic manipulator has an effector unit equipped with a six degrees-of-freedom (6-DOF or 6-axes) force/torque sensor. The effector unit is configured for holding a minimally invasive instrument mounted thereto. In normal use, a first end of the instrument is mounted to the effector unit of the robotic arm and the opposite, second end of the instrument (e.g. the instrument tip) is located beyond an external fulcrum (pivot point kinematic constraint) that limits the instrument in motion. In general, the fulcrum is located within an access port (e.g. the trocar) installed at an incision in the body of a patient, e.g. in the abdominal wall. When an instrument or endoscope is mounted to the robotic arm and inserted through the trocar, a position of the fulcrum along the length of the instrument is determined by the system in a step that will be referred to herein as the “Set Fulcrum” step.

Some surgical robotic systems are configured to support and maneuver rigid endoscopes of a variety of lengths. For example, arms of the Senhance Surgical System marketed by Asensus Surgical, Inc. can receive and manipulate a variety of off-the-shelf endoscopes that are offered in a variety of lengths (e.g. 300 mm and 450 mm). It is important that the length of the endoscope be known to the robotic controller to ensure proper and accurate maneuvering of the endoscope within the body cavity.

This application describes detection of an endoscope length using an automated homing routine during the “Set Fulcrum” process described above with respect to US 2010/0094312. Once the actual endoscope length is identified, the system can control motion of the endoscope and any relevant calculations based on the known length of the endoscope. The ensures safe control of the position and orientation of the endoscope.

Although the inventions described herein may be used on a variety of systems that maneuver an endoscope that extends through an incision or natural orifice into a body cavity, the embodiments will be described with reference to a system of the type shown in FIG. 1. In the illustrated system, a surgeon console 12 has two input devices such as handles 17, 18 that the surgeon selectively assigns to two of the robotic manipulators 13, 14, 15, allowing surgeon control of two of the surgical instruments 10a, 10b, and 10c disposed at the working site at any given time. To control a third one of the instruments disposed at the working site, one of the two handles 17, 18 is operatively disengaged from one of the initial two instruments and then operatively paired with the third instrument. A fourth robotic manipulator, not shown in FIG. 1, may supports and maneuvers an additional instrument.

One of the instruments 10a, 10b, 10c is a laparoscopic camera that captures images for display on a display 23 at the surgeon console 12. The camera may be moved by its corresponding robotic manipulator based on input from an eye tracker 21, one of the handles 17, 18, other user input (e.g. a touch screen on the display 23), or it may move automatically in response to certain conditions or events within the body cavity, such as in a mode by which computer vision or other input is used to automatically track the other instruments as they move within the body cavity.

As described, each robotic manipulator has an effector unit 35 equipped with a six degrees-of-freedom (6-DOF or 6-axes) force/torque sensor similar to that described in US 2010/0094312. The input devices at the console may be equipped to provide the surgeon with tactile feedback generated in response to feedback from the sensor, allowing the surgeon to feel on the input devices 17, 18 forces representing the forces exerted by the instruments on the patient's tissues.

A control unit 30 is operationally connected to the robotic arms and to the user interface. The control unit receives user input from the input devices corresponding to the desired movement of the surgical instruments, and the robotic arms are caused to manipulate the surgical instruments accordingly.

In systems configured to support and maneuver rigid scopes of a variety of lengths, an adapter 36 (FIG. 1B) may be used to mechanically mount the scope 10b to the manipulator. The scope is removably coupled to the adapter 36 (FIG. 1B) and the shaft of the scope is inserted into the body cavity of a patient via a trocar T (FIG. 1C). The adapter/scope assembly is then mounted to the effector unit 35 of the manipulator (FIG. 1D). So that the scope can be accurately maneuvered within the body cavity, input is given to the robotic controller specifying the length of the scope that is in use. This input may be given using an input device at a user interface at the surgeon console (e.g. using a computer touch screen, keyboard, mouse, voice command, manipulation of the input devices 17, 18 to control a mouse cursor on the display, etc.). Alternatively, the system may be configured to receive the input from the adapter, such as by reading data from an RFID tag, EEPROM, etc. The system and method described in this application is useful for verifying the scope length input to the system in this manner to ensure the proper length is taken into account when the robotic controller is controlling motion of the manipulator in the body cavity.

### BRIEF DESCRIPTION OF THE DRAWINGS

FIG. 1A is a perspective view of a prior art robot assisted surgical system;

FIG. 1B shows a scope being mounted to a scope adapter and connected with a light cable;

FIG. 1C shows the scope being inserted through a trocar into a body cavity of a patient;

FIG. 1D shows the adapter being mounted to the manipulator;

FIG. 2 is a block diagram schematically illustrating components of the disclosed system;

FIG. 3 is a flow chart schematically illustrating steps of a method for determining scope length using computer vision;

FIG. 4 is a flow chart schematically illustrating steps of a method for controlling operation of a manipulator arm following measurement of scope length.

### DETAILED DESCRIPTION

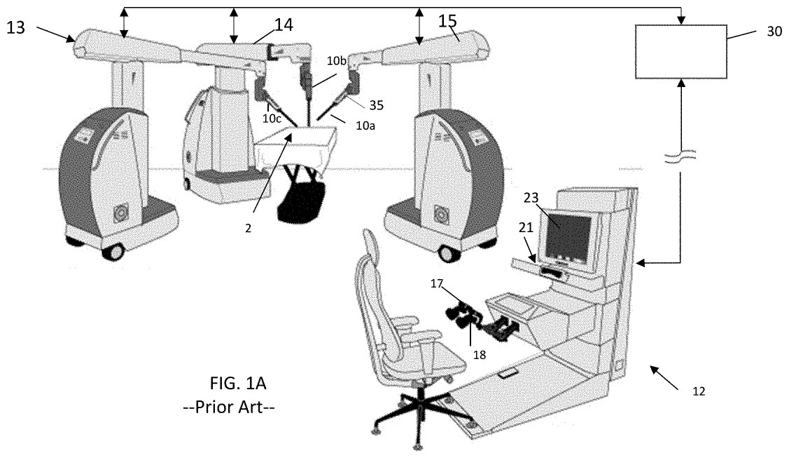

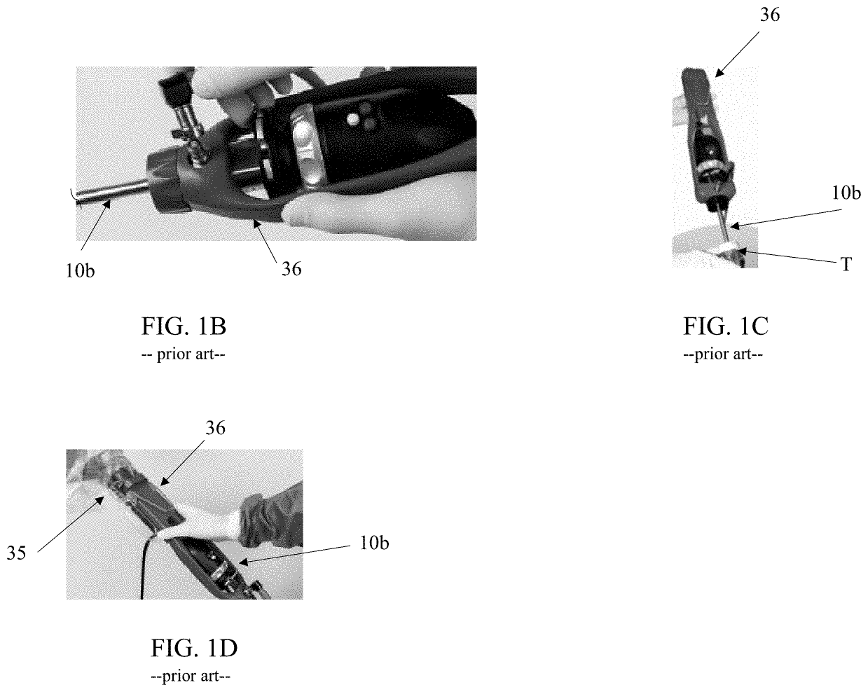

Referring to FIG. 2, a system for determining scope length include the robotic manipulator 14 that supports the scope 10b. The scope 10b captures images within the body cavity within which it is positioned, and those images are displayed on an image display 23, such as the display at the surgeon console (FIG. 1A). One or more processors 30 controls motion of the manipulator. The one or more processors 30 are also programmed with instructions for endoscope length detection which, when executed, carry out the following:
receive image data captured by the scope while causing the manipulator to retract the scope within the body cavity
determine when the distal tip of the trocar T is visible in the image data; and
receive kinematic data from the manipulator arm, and
determine the length of the endoscope using the kinematic data.

The one or more processors may provide other functions as well. For example they might also receive user input from the input devices corresponding to the desired movement of the scope once surgery has begun in the body cavity, and the robotic manipulator is caused to manipulate the surgical instruments accordingly. As discussed, the manipulator may also be controlled using a control algorithm according to which image processing is used to track and cause the scope to follow one of the other surgical instruments being used during surgery so as to maintain that instrument within the visual field displayed on the display 23.

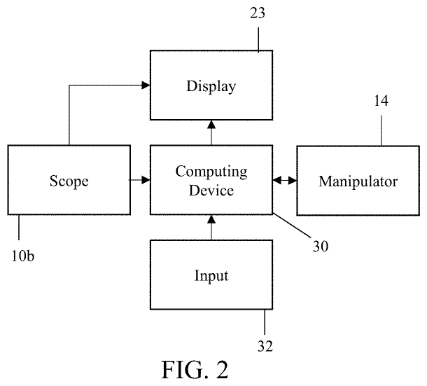

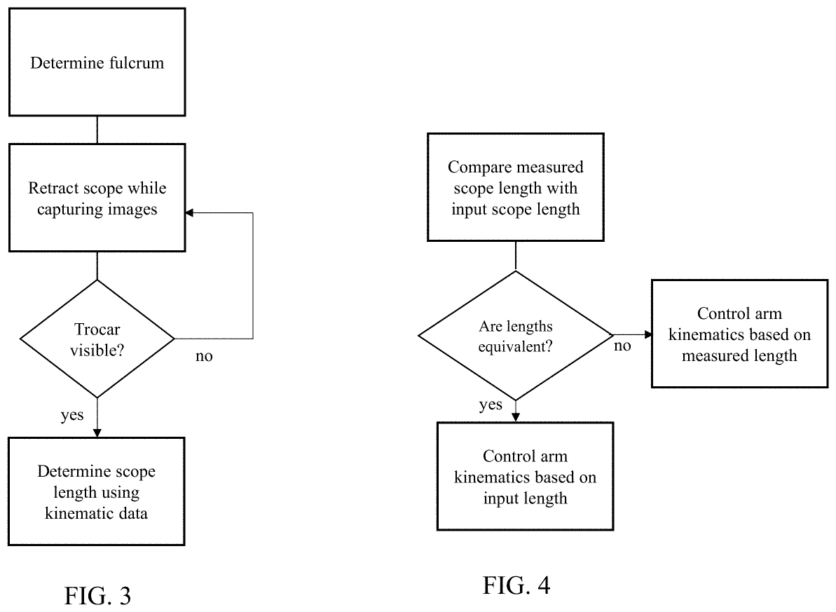

A basic implementation of the automated homing routine for endoscope length detection is described as follows and depicted schematically in FIG. 3. During the Set Fulcrum process for the robotic arm to which the endoscope is mounted, the bedside scrubbed OR staff will place the endoscope into the trocar T positioned through an incision in the body cavity and mount it to the manipulator, and the system will determine the position of the fulcrum as described in US 2010/0094312. After the fulcrum is set, the homing routine will begin. During the homing routine, the endoscope is actively capturing images. The arm will retract the endoscope from the trocar until the end of the trocar is just visible in the endoscope view. Image processing is applied to the images captured by the endoscope and is used to identify the end of the trocar in the captured images. When the end of the trocar is recognized using image processing, the system uses kinematic information from the arm to record the distance between the distal end of the endoscope and the fulcrum.

What happens next is dependent on other features of the system. In general, the measured length is compared with the scope length that was input to the system. If the system is one in which the endoscope is mounted to the robotic arm using an adapter that is equipped to communicate information regarding the scope (or other instrument mounted using the adaptor) to the system (e.g. using an RFID tag read by a sensor on the robotic arm, bar code or QR code read by a reader on the arm etc.) the determined distance may be checked against the endoscope length stored in/on the adapter (e.g. on the adapter's RFID chip). If a user entered the scope length information using an input device, the determined distance is checked against that length. If there is a sufficiently large mismatch in the measured length and the expected length of the endoscope, it can be determined that the user did not correctly enter the scope length or select the correct adapter (which would have correctly identified the endoscope length) for the scope mounted to the manipulator. The system can then ignore the input length or information stored in the adapter's RFID chip and instead use the appropriate endoscope control information (length, mass, center of mass, etc.) based on the measured length. In other embodiments, for example where the adapter does not communicate instrument/endoscope parameters to the system, the system could rely on the endoscope control information stored in the system's memory for endoscopes of that length.

If desired, following the homing routine, the system may cause the robotic arm to move the endoscope back to its initial position and orientation (the position before the homing routine was conducted). Returning the endoscope to its initial position would likely be preferred over leaving the endoscope at the distal end of the trocar and requiring either the bedside scrubbed OR staff or the surgeon to return the endoscope to its initial position. The endoscope could be safely moved in an automated process by following the same path that was taken to reach the end of the trocar (in reverse) while preventing movement of other arms to minimize risk of collision.

In a first alternative embodiment, the manipulator arm may be used to sweep the endoscope within the body cavity, relative to the fulcrum. In this embodiment, length is calculated based on how much sweep occurs outside of the trocar. In addition, or as an alternative method to those described above, the endoscope may be removed from the trocar, and the force/torque sensor in the arm may be used to measure the weight of the endoscope. In this embodiment, the weight of the endoscope is compared with weights of endoscopes of various lengths in the system memory, and the system determines which length endoscope is being used.

We claim:

1. A method of determining a length of an endoscope carried by a robotic arm, the method comprising:
mounting the endoscope to a robotic arm;
inserting the distal end of the endoscope through a trocar into a body cavity;
determining the position of a fulcrum for movement of the endoscope at the trocar;
while capturing images using the endoscope, withdrawing the distal end of the endoscope intro the trocar'
using image processing, recording the position of the endoscope when the trocar becomes visible, and determining the length of the endoscope based on the recorded position.
